(19) United States
(12) Patent Application Publication
(10) Pub. No.: US 2020/0054220 A1
(43) Pub. Date: Feb. 20, 2020

(54) Physiological Parameter Recording System and Method Thereof

(71) Applicant: EBM TECHNOLOGIES INCORPORATED, Taipei City (TW)

(72) Inventor: William Pan, Taipei City (TW)

(21) Appl. No.: 16/239,560

(22) Filed: Jan. 4, 2019

Related U.S. Application Data
(60) Provisional application No. 62/718,889, filed on Aug. 14, 2018.

(30) Foreign Application Priority Data
Oct. 22, 2018 (TW) 107137187

(51) Int. Cl. A61B 5/0245; A61B 5/00; A61B 5/0205; A61B 5/024

(52) U.S. Cl. CPC A61B 5/0245; A61B 5/0015; A61B 2560/0271; A61B 5/02438; A61B 5/0205

(57) ABSTRACT

A physiological parameter recording system includes a first measurement equipment configured to generate a first physiological parameter, a second measurement equipment configured to generate a second physiological parameter, a recording device installed with a physiological parameter recording program configured to execute the following steps: reading the first physiological parameter by connecting to the first physiological parameter through a digital transmission function, reading the second physiological parameter by a user input interface, displaying the first and second physiological parameters, confirming and storing the first and second physiological parameters after the user input interface has been locked according to a lock signal, and generating and displaying a quick response code according to the first and second physiological parameters, and a cloud database configured to receive the quick response code from the recording device through the digital transmission function.

### Cross Reference To Related Applications

This application claims the benefit of U.S. Provisional Application No. 62/718,889, filed on Aug. 14, 2018, the contents of which are incorporated herein.

### BACKGROUND OF THE INVENTION

### 1. Field of the Invention

The present invention relates to a recording system and method thereof, and more particularly, to a physiological parameter recording system and method thereof.

### 2. Description of the Prior Art

Regarding the method of recording physiological parameters, it is mostly done by manual transcription and filing. For example, a physician and a nurse of a hospital and a healthcare giver of a health care center may utilize a medical equipment to measure physiological parameters (e.g., body temperature, blood pressure, heart rate, respiratory rate, blood oxygen level, glucose, and so on) of a patient, or query activities and feelings of the patient (e.g., sleeping time, a number of defecation, a pain index, and so on). Then, the measured physiological parameters, activities and feelings are written into papers to input to an electronic database.

However, above mentioned routines are time wasting, and may cause typographic errors to medical record due to manual work. Therefore, there is a need to provide a physiological parameter recording system and method thereof capable of automatic recording and integration, to improve convenience and correctness.

### SUMMARY OF THE INVENTION

It is therefore an objective of the present invention to provide a physiological parameter recording system and method thereof capable of automatic recording and integration.

The present invention discloses a physiological parameter recording system, includes a first measurement equipment, configured to generate a first physiological parameter, and transmit the first physiological parameter through a digital transmission function; a second measurement equipment, configured to generate a second physiological parameter; a recording device, installed with a physiological parameter recording program to execute the following steps: connecting to the first measurement equipment through the digital transmission function to read the first physiological parameter; reading the second physiological parameter through an user input interface; displaying the first physiological parameter and the second physiological parameter; confirming and storing the first physiological parameter and the second physiological parameter according to a lock signal, after the user input interface is locked; and generating and displaying a first quick response code according to the first physiological parameter and the second physiological parameter; and a cloud database, connected to the recording device through the digital transmission function to receive the first quick response code.

The present invention discloses a method of recording physiological parameter, for a recording device of a physiological parameter recording system includes the recording device through digital transmission function, connecting to a first measurement equipment of the physiological parameter recording system, to read a first physiological parameter of the first measurement equipment generate; the recording device through a user input interface, reading a second physiological parameter generated by a second measurement equipment the physiological parameter recording system; the recording device displaying the first physiological parameter and the second physiological parameter; the recording device confirming and storing the first physiological parameter and the second physiological parameter according to a lock signal after the user input interface is locked; the recording device generating and displaying a first quick response code according to the first physiological parameter and the second physiological parameter; and the recording device through digital transmission function, connecting to a cloud database of the physiological parameter recording system to transmit the first quick response code.

In the physiological parameter recording system of the present invention, the recording device may connect to the multiple measurement equipment 10 through wireless connection to automatically obtain multiple physiological parameters (or manually input the physiological parameter by the operator), then generate the corresponding QR code according to the stored physiological parameters, and finally upload the QR code (i.e., multiple physiological parameters) to the cloud database through wireless network. As a result, the physiological parameter recording system of the present invention may reduce manual transcription and filing, reduce a probability of typographic errors, so as to realize automatic recording and integration to multiple physiological parameters.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram of a physiological parameter recording system according to an embodiment of the present invention.

FIG. 2 is a flowchart of a physiological parameter recording process according to an embodiment of the present invention.

FIG. 3 to FIG. 10 illustrates operations of a recording device according to an embodiment of the present invention.

### DETAILED DESCRIPTION

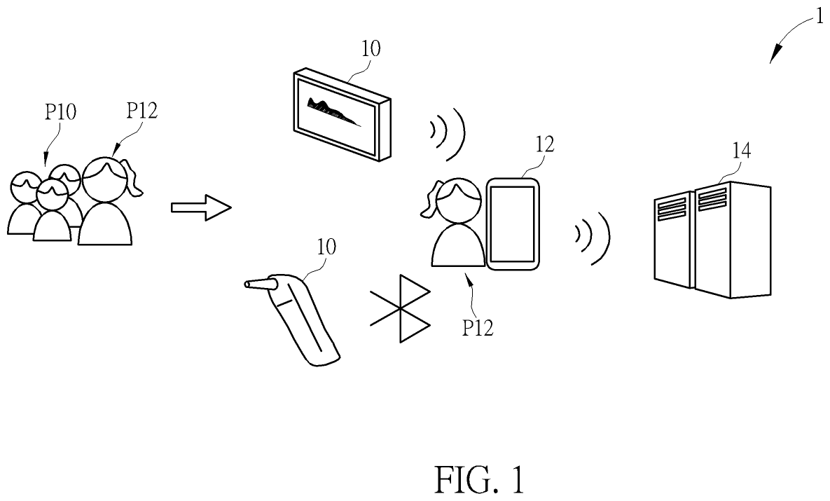

FIG. 1 is a schematic diagram of a physiological parameter recording system 1 according to an embodiment of the present invention. The physiological parameter recording system 1 may be utilized in any organizations and institutes which requires measurement and record of physiological parameter, such as:

(1) Examine room of a hospital, a clinic, a ward;

(2) Health care institute, long-term health care center;

(3) Medical research school and institute;

(4) Healthcare, beauty salon, rehabilitation center;

(5) Seminar, research and conference meeting;

(6) Household care.

The physiological parameter recording system 1 includes multiple measurement equipment 10, a recording device 12, and a cloud database 14. The measurement equipment 10 may be medical equipment for measuring physiological parameters, such as a thermometer, sphygmomanometer, pulse oximeter, glucose meters, a vital signs monitor, a body weight machine, and so on. The recording device 12 may a mobile device equipped with a wireless communication function (such as Wifi, third generation wireless communication, fourth generation wireless communication, long-term evolution, Bluetooth, near field communication, and so on), to allow user to download and install an application from the Internet, and perform operations on a touch screen of the mobile device such as a smart phone or a tablet computer. The cloud database 14 may be a medical information integration system.

The physiological parameter recording system 1 is configured to assist an operator P12 to respectively record physiological parameters for multiple subjects P10, wherein the operator P12 may be a physician, a nurse or a care worker, and the subject P10 may be a patient or an ordinary people. The physiological parameter a height, a weight, a head circumference, a neck circumference, abreast circumference, a waist circumference, a hip circumference, a heart rate, a systolic blood pressure, a diastolic blood pressure, blood oxygen level, a blood glucose, a body temperature, a respiratory rate, a pain index, a urine volume or a number of defecation, which is not limited.

Specifically, the recording device 12 may connect to multiple measurement equipment 10, the operator P12 may operate the measurement equipment 10 to obtain the physiological parameter of the subject P10; then, through digital transmission function (e.g., Bluetooth, near field communication, Wifi, and so on) of the measurement equipment 10, the recording device 12 may gather the physiological parameter measured by the measurement equipment 10. When the measurement equipment 10 does not equip with the digital transmission function, the operator P12 may input the physiological parameter measured by the measurement equipment 10 to the recording device 12 through a user input interface (e.g., keyboard). When all the measurements of the physiological parameters have been finished, the recording device 12 may confirm and store all the physiological parameters according to a lock signal after the user input interface has been locked, and generate a corresponding QR (Quick Response) code. As a result, the recording device 12 may import the physiological parameters to a personal health file or a webpage record of subject P10 through the QR code, so as to store the physiological parameters in the cloud database 14.

In brief, in the physiological parameter recording system 1, the recording device 12 may connect to the multiple measurement equipment 10 to automatically obtain multiple physiological parameters (or manually input the physiological parameter by the operator), then generate the corresponding QR code according to the stored physiological parameters, and finally upload the QR code (i.e., multiple physiological parameters) to the cloud database 14 through wireless network. As a result, the physiological parameter recording system 1 of the present invention may reduce manual transcription and filing, reduce a probability of typographic errors, so as to realize automatic recording and integration to multiple physiological parameters.

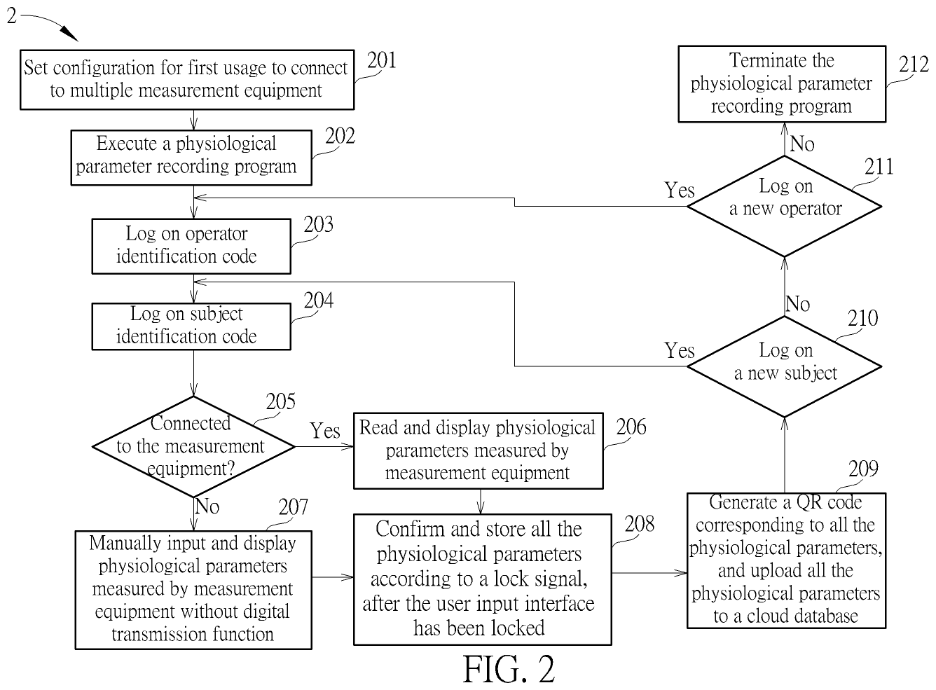

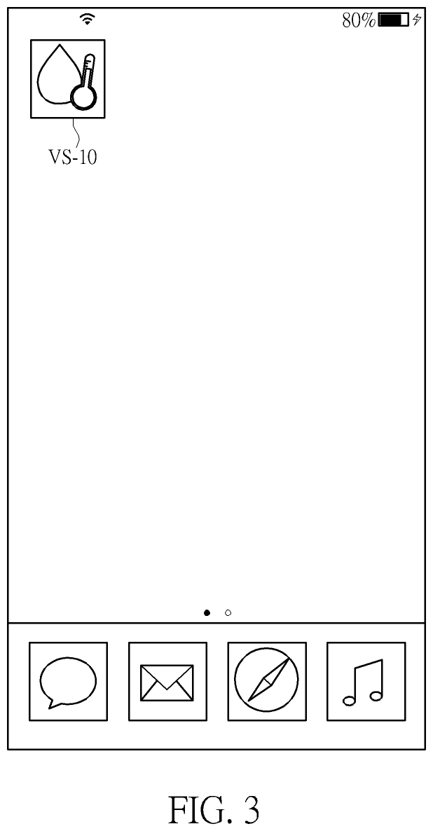

FIG. 2 is a flowchart of a physiological parameter recording process 2 according to an embodiment of the present invention, and FIG. 3 to FIG. 10 illustrates operations of the recording device 12 according to an embodiment of the present invention. The operator P12 may operate the recording device 12 by executing an application according to steps of the physiological parameter recording process 2, so as to realize automatic recording and integration to multiple physiological parameters. The physiological parameter recording process 2 includes the following steps.

Step 201: Set configuration for first usage to connect to multiple measurement equipment.

Step 202: Execute a physiological parameter recording program.

Step 203: Log on operator identification code.

Step 204: Log on subject identification code.

Step 205: Whether connected to the measurement equipment? Go to Step 206 if yes; go to Step 207 if no.

Step 206: Read and display physiological parameters measured by measurement equipment. Go to Step 208.

Step 207: Manually input and display physiological parameters measured by measurement equipment without digital transmission function.

Step 208: Confirm and store all the physiological parameters according to a lock signal, after the user input interface has been locked.

Step 209: Generate a QR code corresponding to all the physiological parameters, and upload all the physiological parameters to a cloud database.

Step 210: Determine whether to log on a new subject? Return to Step 204 if yes; go to Step 211 if no.

Step 211: Determine whether to log on a new operator? Return to Step 203 if yes; go to Step 212 if no.

Step 212: Terminate the physiological parameter recording program.

In Step 201, the operator P12 may set configuration for first usage to connect to the multiple measurement equipment 10, for example, turn on the wireless communication function (e.g., WiFi, near field communication, Bluetooth) of the recording device 12 to connect to the multiple measurement equipment 10; download and install the physiological parameter recording program. In Step 202, as shown in FIG. 3, the operator P12 may download and install a physiological parameter recording program VS-10 to execute the physiological parameter recording program VS-10.

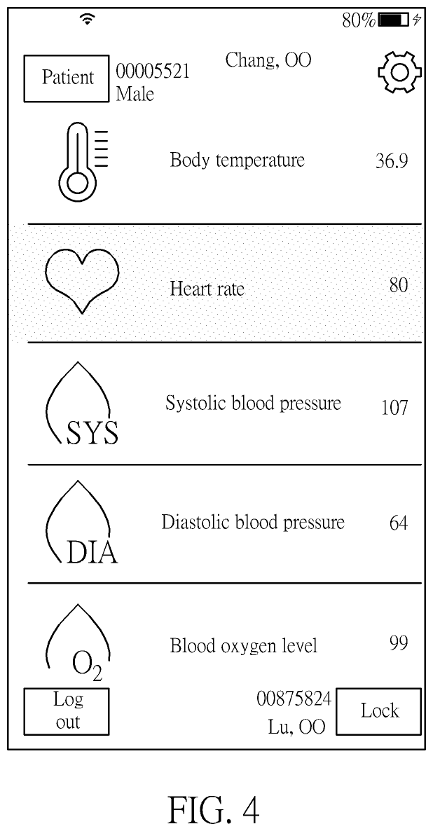

In Step 203, the operator P12 may log on and display an operator identification code though scanning a QR code or a bar code or manually input, as shown in FIG. 4 which illustrates an operator identification code “000875824” and an operator name “Lu, 00”. In Step 204, the operator P12 may log on and display a subject identification code through scanning a QR code or a bar code or manually input, as shown in FIG. 4 which illustrates a subject identification code “00005521” and a subject name “Chang, 00”. Note that scanning the QR code or bar code to log on the operator identification code and the subject identification code may avoid typographic errors and speed up recording process.

In Step 205 to Step 206, when the recording device 12 is connected to the measurement equipment 10, the recording device 12 may read and display the physiological parameter measured by measurement equipment 10. As shown in FIG. 4, the recording device 12 may connect to a forehead thermometer or an ear thermometer to measure a body temperature “36.9 Celsius degrees”. The recording device 12 may connect to a vital sign monitor to measure a heart rate “80 times/minute”, a systolic blood pressure “107 mm/Hg, and a diastolic blood pressure “64 mm/Hg”. The recording device 12 may connect to a pulse oximeter to measure a blood oxygen level is “99%”.

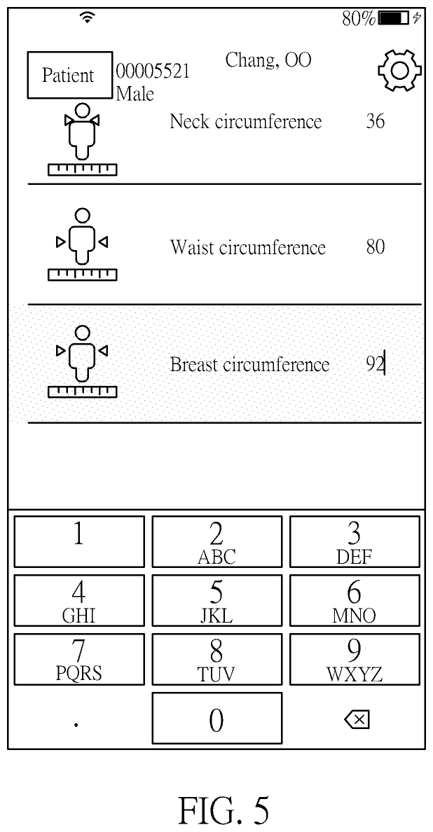

In Step 205 to Step 207, when the recording device 12 is unable to connect to the measurement equipment 10, the operator P12 may manually perform measurement to the subject P10 and manually input the physiological parameter measured by the measurement equipment 10 without digital transmission function to the recording device 12. As shown in FIG. 5, the operator P12 may use tape measure to perform measurement to the subject P10, and input a neck circumference “36 cms”, a waist circumference “80 cms”, and a breast circumference “92 cms” of the subject P10 to the recording device 12 through a user input interface (e.g., keyboard). In other words, the recording device 12 may read the physiological parameter through a user input interface.

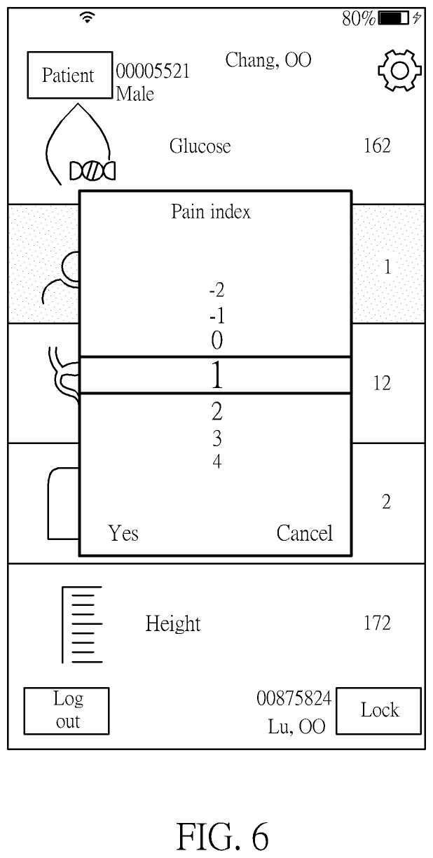

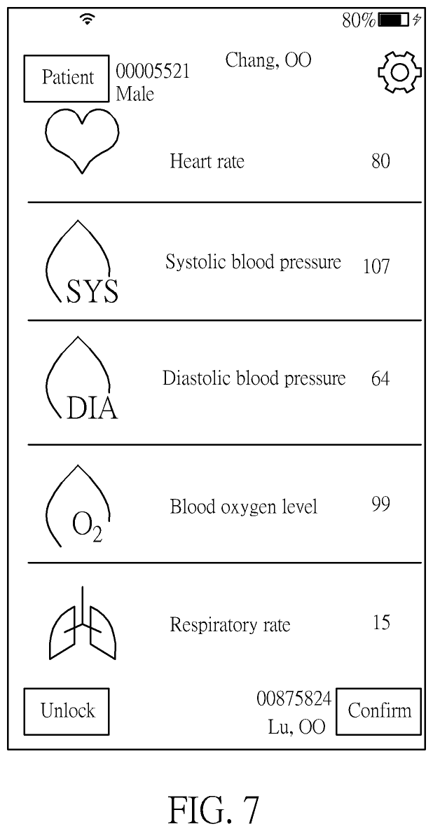

As shown in FIG. 6, the operator P12 may query a pain index of the subject P10, and select a pain index “level 1” from a popup window of recording device 12. In one embodiment, as shown in FIG. 6, the recording device 12 may display a “lock” selection configured to lock the inputted physiological parameters, which may avoid typographic errors due to disoperation. As shown in FIG.7, when the stored physiological parameters are locked, the operator P12 may select an “unlock” selection displayed by the recording device 12, and then perform measurement again to modify the physiological parameter.

In Step 208, when the operator P12 has confirmed all the physiological parameters are correct, the operator P12 may select a “confirm” selection to store all the physiological parameters after the “lock” selection is selected to lock the user input interface, so as to finish a cycle of recording physiological parameter for the sole subject P10. In other words, the recording device 12 may confirm and store all the physiological parameters according to a lock signal after the user input interface is locked.

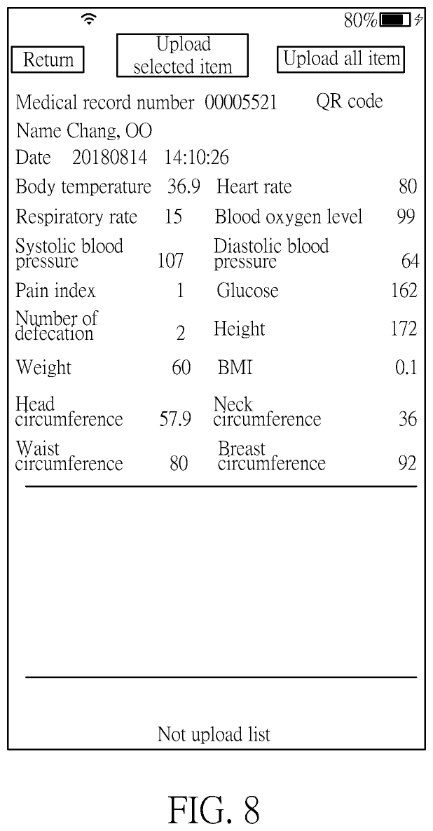

As shown in FIG. 8, after finishing the measurement, the recording device 12 may display the record of the physiological parameters in a screen. In one embodiment, the operator P12 may select a “upload selected item” selection or a “upload all items” selection according to practical requirements to improve flexibility. In addition, since the recording device 12 may store the recorded data (i.e., the physiological parameters), and then upload data through the Internet, the recorded data may be protected from bad network environment due to temporary network failure.

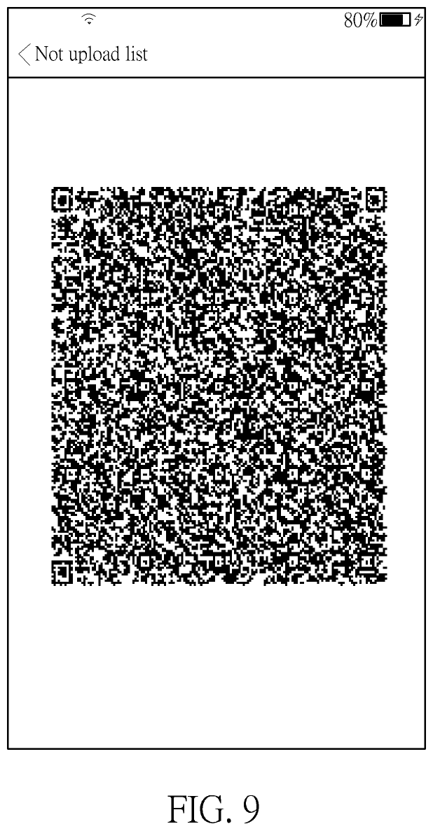

In Step 209, as shown in FIG. 9, the recording device 12 may generate a QR code corresponding to the physiological parameters, to upload the physiological parameter to the cloud database. In one embodiment, the recording device 12 may record a recording timestamp and an upload timestamp of the physiological parameters (or, the QR code), which is advantageous to the operator P12 to manage the physiological parameters of the subject P10. In practice, when the operator P12 has returned to a work station, the operator P12 may scan the identification code (e.g., a two dimensional bar code) of the subject P10 and the QR code corresponding to the physiological parameters, so as to integrate the recorded data in the cloud database (e.g., a hospital information system) and avoid additional time for manual transcription.

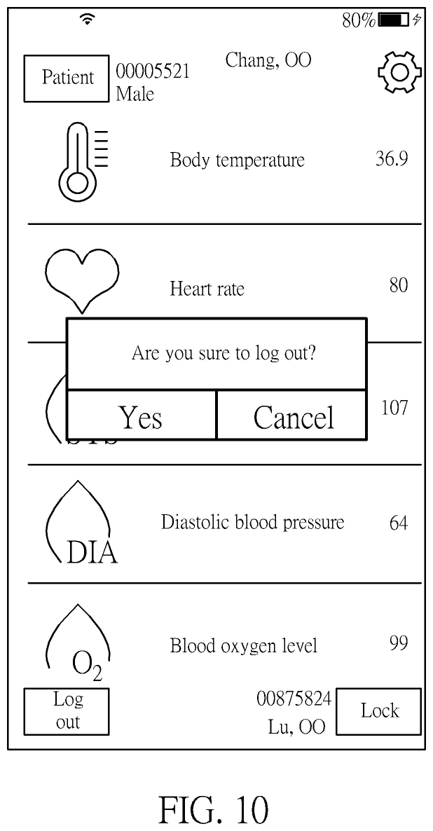

In Step 210, when there is a need to log on a new subject, the operator P12 may log on a subject identification code through scanning a QR code or a bar code, or manually input method to perform Step 204-Step 209 again. In Step 211, when there is a need to log on a new operator, the new operator P12 may log on a new operator identification code through scanning a QR code or a bar code, or manually input method to perform Step 203-Step 209. In Step 212, as shown in FIG. 10, when there is no need to log on the new subject and the new operator, the physiological parameter recording program is terminated to end the physiological parameter recording process 2.

To sum up, in the physiological parameter recording system of the present invention, the recording device may connect to the multiple measurement equipment 10 through wireless connection to automatically obtain multiple physiological parameters (or manually input the physiological parameter by the operator), then generate the corresponding QR code according to the stored physiological parameters, and finally upload the QR code (i.e., multiple physiological parameters) to the cloud database through wireless network. As a result, the physiological parameter recording system of the present invention may reduce manual transcription and filing, reduce a probability of typographic errors, so as to realize automatic recording and integration to multiple physiological parameters.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A physiological parameter recording system, comprising:
a first measurement equipment, configured to generate a first physiological parameter, and transmit the first physiological parameter through a digital transmission function;
a second measurement equipment, configured to generate a second physiological parameter;
a recording device, installed with a physiological parameter recording program to execute the following steps:
connecting to the first measurement equipment through the digital transmission function to read the first physiological parameter;
reading the second physiological parameter through an user input interface;
displaying the first physiological parameter and the second physiological parameter;
confirming and storing the first physiological parameter and the second physiological parameter according to a lock signal, after the user input interface is locked; and
generating and displaying a first quick response code according to the first physiological parameter and the second physiological parameter; and
a cloud database, connected to the recording device through the digital transmission function to receive the first quick response code.

2. The physiological parameter recording system of claim 1, wherein the physiological parameter recording program controls the recording device to execute the following steps:
through scanning a second quick response code or a first bar code, or through the user input interface to log on and display a first operator identification code; and
through scanning a third quick response code or a second bar code, or through the user input interface to log on and display a first subject identification code.

3. The physiological parameter recording system of claim 1, wherein the physiological parameter recording program controls the recording device to execute the following steps:
when determining a need to log on a new subject, through scanning a fourth quick response code or the third bar code, or through the user input interface log on and display a second subject identification code; and
when determining no need to log on the new subject, determining whether to log on a new operator, comprising:
when determining a need to log on the new operator, through scanning a fifth quick response code or a fourth bar code, or through the user input interface to log on and display a second operator identification code; and
when determining no need to log on the new operator, terminates the physiological parameter recording program.

4. The physiological parameter recording system of claim 1, wherein the recording device through the digital transmission function being Wifi, Bluetooth or near field communication to connect to the first measurement equipment, and the cloud database through the digital transmission function being Wifi, third generation wireless communication, fourth generation wireless communication, long term evolution, blue tooth, or near field communication to connect to the recording device.

5. The physiological parameter recording system of claim 1, wherein the first measurement equipment and the second measurement equipment is one of a thermometer, sphygmomanometer, pulse oximeter, glucose meters, a vital signs monitor, a body weight machine, and the first physiological parameter and the second physiological parameter is one of a height, a weight, a head circumference, a neck circumference, a breast circumference, a waist circumference, a hip circumference, a heart rate, a systolic blood pressure, a diastolic blood pressure, blood oxygen level, a blood glucose, a body temperature, a respiratory rate, a pain index, a urine volume and a number of defecation.

6. A method of recording physiological parameter, for a recording device of a physiological parameter recording system, comprising:
the recording device through digital transmission function, connecting to a first measurement equipment of the physiological parameter recording system, to read a first physiological parameter of the first measurement equipment generate;
the recording device through a user input interface, reading a second physiological parameter generated by a second measurement equipment the physiological parameter recording system;
the recording device displaying the first physiological parameter and the second physiological parameter;
the recording device confirming and storing the first physiological parameter and the second physiological parameter according to a lock signal after the user input interface is locked;
the recording device generating and displaying a first quick response code according to the first physiological parameter and the second physiological parameter; and
the recording device through digital transmission function, connecting to a cloud database of the physiological parameter recording system to transmit the first quick response code.

7. The physiological parameter recording of claim 6, further comprising:
the recording device through scanning a second quick response code or a first bar code, or through the user input interface to log on and display a first operator identification code; and
the recording device through scanning a third quick response code or a second bar code, or through the user input interface to log on and display a first subject identification code.

8. The physiological parameter recording of claim 6, further:
when determining a need to log on a new subject, through scanning a fourth quick response code or the third bar code, or through the user input interface log on and display a second subject identification code; and
when determining no need to log on the new subject, determining whether to log on a new operator, comprising:
when determining a need to log on the new operator, through scanning a fifth quick response code or a fourth bar code, or through the user input interface to log on and display a second operator identification code; and
when determining no need to log on the new operator, terminates the physiological parameter recording program.

9. The physiological parameter recording of claim 6, wherein the recording device through the digital transmission function being Wifi, Bluetooth or near field communication to connect to the first measurement equipment, and the cloud database through the digital transmission function being Wifi, third generation wireless communication, fourth generation wireless communication, long term evolution, blue tooth, or near field communication to connect to the recording device.

10. The physiological parameter recording of claim 6, wherein the first measurement equipment and the second measurement equipment is one of a thermometer, sphygmomanometer, pulse oximeter, glucose meters, a vital signs monitor, a body weight machine, and the first physiological parameter and the second physiological parameter is one of a height, a weight, a head circumference, a neck circumference, a breast circumference, a waist circumference, a hip circumference, a heart rate, a systolic blood pressure, a diastolic blood pressure, blood oxygen level, a blood glucose, a body temperature, a respiratory rate, a pain index, a urine volume and a number of defecation.
